(19) United States
(12) Patent Application Publication
(10) Pub. No.: US 2008/0039897 A1
(43) Pub. Date: Feb. 14, 2008

(54) Trans-Septal Left Ventricular Pressure Measurement

(76) Inventors: Stanley E. Kluge, St. Paul, MN (US); Scott Thomas Mazar, Woodbury, MN (US); Eric N. Rudie, Maple Grove, MN (US); Lynn M. Zwiers, Lino Lakes, MN (US)

Correspondence Address: FISH & RICHARDSON P.C., P.O. BOX 1022, MINNEAPOLIS, MN, 55440-1022, US

(21) Appl. No.: 11/836,592

(22) Filed: Aug. 9, 2007

Related U.S. Application Data
(60) Provisional application No. 60/837,352, filed on Aug. 10, 2006.

(51) Int. Cl. A61B 5/05; A61N 1/05

(52) U.S. Cl. 607/17; 600/411

(57) ABSTRACT

A pressure sensing device includes a body portion, a pressure transmitting port, and an electrical lead. The body portion includes transducing electronics within a housing that is shaped about a longitudinal axis. The housing has a coating thereon that promotes tissue growth to anchor the housing within a ventricular septum. The pressure transmitting port is located at a distal longitudinal end of the body portion such that a ventricle pressure being sensed is transmitted through the port and to the transducing electronics when the body portion is anchored in the ventricular septum. The electrical lead is connected to the transducing electronics and exits from a proximal longitudinal end of the body portion.

### CROSS-REFERENCE TO RELATED APPLICATIONS

The present application claims the benefit of U.S. Provisional Patent Application No. 60/837,352, filed Aug. 10, 2006, the entire disclosure of which is incorporated herein by reference.

### TECHNICAL FIELD

This application relates to a trans-septal ventricular pressure measurement device and a method of implanting the device.

### BACKGROUND

Pressure measurement devices can be used to sense numerous internal body pressures in humans and animals. Examples of pressures that can be sensed include pulmonary pressure, venous pressure, left ventricle pressure, intracranial pressure, and bladder pressure. These measurements provide an important tool for medical research and clinical diagnosis.

Congestive Heart Failure (CHF) is an end-stage chronic condition resulting from the heart's inability to pump sufficient blood, and is a significant factor in morbidity, mortality and health care expenditure. There are a variety of underlying conditions that can lead to CHF, and a variety of therapeutic approaches targeting such conditions. The selection of the therapeutic approach, and the parameters of the particular therapeutic approach selected, is a function of the underlying condition and the degree to which it affects the heart's ability to pump blood. Endocardial pressure, particularly left ventricular (LV) pressure, is a good indicator of the heart's ability to pump blood and the effectiveness of any given therapy.

Studies have shown that patients with moderate to severe CHF can benefit from Cardiac resynchronization therapy (CRT). CRT devices are similar to conventional pacemakers, except that in addition to a lead for pacing the right ventricle, a CRT device includes a lead for pacing the left ventricle. Left ventricular leads can be placed intravascularly using a coronary sinus lead, or surgically using an epicardial lead. An example of a commercially available CRT device is the InSync® system from Medtronic. However, such CRT systems do not have the ability to measure LV pressure.

### SUMMARY

A pressure sensing device is described that includes a body portion, a pressure transmitting port, and an electrical lead. The body portion includes transducing electronics within a housing that is shaped about a longitudinal axis. The housing has a coating thereon that promotes tissue growth to anchor the housing within a ventricular septum. The pressure transmitting port is located at a distal longitudinal end of the body portion such that a ventricle pressure being sensed is transmitted through the port and to the transducing electronics when the body portion is anchored in the ventricular septum. The electrical lead is connected to the transducing electronics and exits from a proximal longitudinal end of the body portion.

In some embodiments, the coating can include pores. For example, the coating can promote tissue ingrowth of the ventricular septum into the pores to anchor the body portion in the ventricular septum. In some embodiments, the coating can include expanded polytetrafluoroethylene and/or polyethylene terephthalate.

A method of implanting the pressure sensing device is also described. The method includes inserting the pressure sensing device through a ventricular septum to sense a pressure in a left ventricle. Inserting the pressure sensing device includes positioning the body portion in the ventricular septum and the port in the left ventricle.

In some embodiments, the body portion can be anchored in the ventricular septum by frictional engagement between the coating and the ventricular septum.

In some embodiments, the method can include forming a passage in the ventricular septum. Inserting the pressure sensing device can include passing the pressure sensing device through the passage. In some embodiments, the passage can have a first diameter and the housing can have a second diameter greater than the first diameter. Inserting the body portion having the larger diameter through the passage can result in the final dilatation of the passage.

In some embodiments, the method can further include inserting an introducing apparatus into a vein. The introducing apparatus can include an introducer, a sheath disposed at least partially in the introducer, a centering tube disposed at least partially in the sheath, and a needle disposed at least partially in the centering tube. The centering tube can center the needle with respect to the sheath. The method can also include advancing the introducing apparatus to a right ventricle. A distal end of the introducing apparatus can be placed against the ventricular septum. The needle can be extended through the ventricular septum into a left ventricle for initial registration. The method can include extending the sheath partially into the ventricular septum for registration and removing the needle and the centering tube and leaving the sheath in place to maintain registration. The pressure sensing device can be passed through an interior of the sheath to insert the pressure sensing device through the ventricular septum. The sheath and the introducer can be removed without dislodging the pressure sensing device. The introducing apparatus can be assembled prior to being inserted into the vein, so that a distal end of the sheath protrudes slightly through a flared distal end of the introducer, and a distal end of the needle extends through a distal end of the centering tube and the distal end of the sheath.

In some embodiments, the method can further include using a pressure measurement at the distal tip of the introducing apparatus to determine a location of the distal tip of the introducing apparatus while advancing the introducing apparatus to the right ventricle based on pressure changes from one location to another location while advancing the introducing apparatus to the right ventricle. The method can also use fluoroscopy to determine a location of the distal tip of the introducing apparatus while advancing the introducing apparatus to the right ventricle.

In some embodiments, the method can include confirming a location of a distal tip of the needle while extending the needle through the ventricular septum into the left ventricle for initial registration and/or confirming a location of the pressure sensing device while inserting the pressure sensing device through the interior of the sheath into the ventricular septum.

In some embodiments, the needle can be a modified Brockenbrough needle. In some embodiments, the centering tube can have an outer diameter substantially equal to an outer diameter of the pressure measurement device and/or an inner diameter substantially equal to an outer diameter of the needle.

The details of one or more implementations are set forth in the accompanying drawings and the description below. Other features, objects, and advantages will be apparent from the description and drawings, and from the claims.

### DESCRIPTION OF DRAWINGS

FIG. 1 is a schematic diagram illustrating an example of a system which communicates with the implantable pressure sensing device, including a home (i.e., local) data collection system (HDCS) and a physician (i.e., remote) data collection system (PDCS).

FIG. 2 is a perspective view of the implantable pressure sensing telemetry device, including a remote sensor assembly (RSA) and telemetry unit (TU), in accordance with an exemplary implementation.

FIG. 3A depicts a perspective view of the RSA, including a body portion having transducing electronics within a housing, a pressure transmitting port as part of a pressure transmission catheter (PTC), and a coating overlying the housing and a portion of the PTC.

FIG. 3B depicts a cross-sectional view of the electronics module.

FIGS. 3C and 4 depict the RSA implanted into a ventricular septum.

FIG. 5 is a photograph of an RSA implanted into a ventricular septum.

FIGS. 6A and 6B depict an introductory apparatus for implanting the RSA into a ventricular septum.

Like reference symbols in the various drawings indicate like elements.

### DETAILED DESCRIPTION

### Brief Description of the System

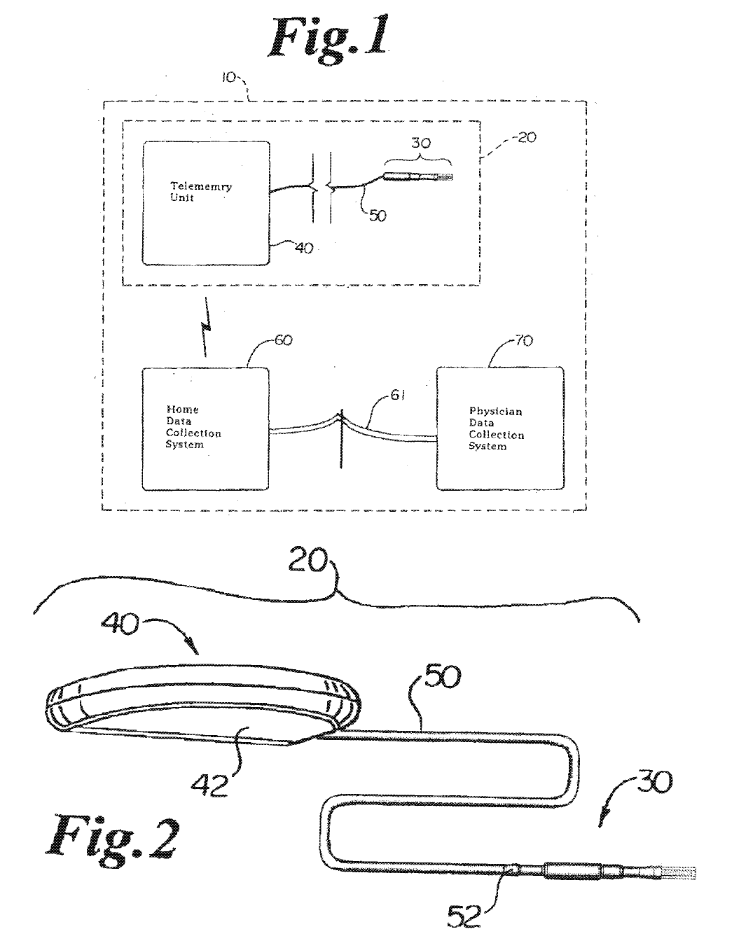

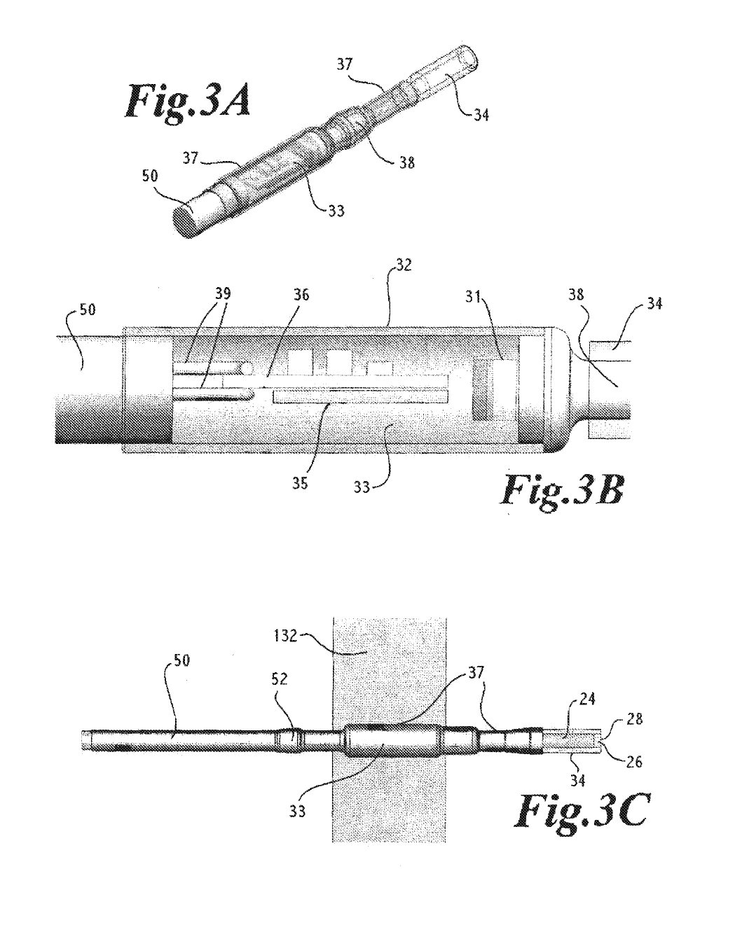

The pressure sensing device, in some implementations, can be part a system 10 for measuring and monitoring endocardial pressure (e.g., LV pressure). An example of the overall system 10 is shown in FIG. 1. The system 10 can include an implantable telemetry device (ITD) 20, shown in FIG. 2, which includes a remote sensor assembly (RSA) 30 for measuring endocardial pressure, connected via a lead 50 to a telemetry unit (TU) 40 for telemetering measured pressure data to a receiver located outside the body. The system 10 can also include a home (i.e., local) data collection system (HDCS) 60 which can receive the telemetry signal, optionally correct for fluctuations in ambient barometric pressure, evaluate the validity of the received signal, and, if the received signal is deemed to be valid, extract parameters from that signal and store the data according to a physician-defined protocol.

The system 10 also includes a physician (i.e., remote) data collection system (PDCS) 70 which can receive the data signal from the HDCS 60 via a telecommunication system 61 (e.g., the Internet). The PDCS 70 receives the data signal, evaluates the validity of the received signal and, if the received signal is deemed to be valid, displays the data, and stores the data according to a physician-defined protocol. With this information, the system 10 can enable the treating physician to monitor endocardial pressure in order to select and/or modify therapies for the patient to better treat diseases such as CHF and its underlying causes.

For example, the system 10 can be used for assessment of pressure changes (e.g., systolic, diastolic, and LV max dP/dt) in the main cardiac pumping chamber (the LV). These pressures are known to fluctuate with clinical status in CHF patients, and can provide key indicators for adjusting treatment regimens. For example, increases in end diastolic pressure, changes in the characteristics of pressure within the diastolic portion of the pressure waveform, and decreases in maximum dP/dt, or increases in minimum dP/dt together suggesting a deteriorating cardiac status. As used herein, LV max dP/dt can refer to the maximum rate of pressure development in the left ventricle. These measurements could be obtained either during clinic visits or from the patient at home, from the proposed device, and stored for physician review. The physician can then promptly adjust treatment. In addition, the system 10 can assist in management of patients when newer forms of device therapy (e.g., multiple-site pacing, ventricular assist as a bridge to recovery, or implantable drugs pumps) are being considered.

It can also be useful to automate or partially automate some level of interaction with the patient. For example, departures from prescribed limits or values for certain patient parameters can be noted automatically and brought to the attention of the physician or patient. The ability to automatically select deteriorating patients from the much larger pool of monitored patients may save a practitioner's time and improve patient care.

The system 10 can create an exception report on a daily basis to create a list of patients requiring special follow-up or care. More specifically, the system 10 can interact with the patient directly and request additional monitoring or compliance with a specific health care regime. The limits which trigger the exception report can be under the control of an attending physician.

More specifically, information received in the clinic by the PDCS 70 from the HDCS 60 can be evaluated and triaged for follow-up by a medical practitioner. Following evaluation of the information received in physician's office or clinic, the system 10 can create an exception report that lists patients to be contacted for follow-up. Patients at home can be monitored using the ITD 20 and HDCS 60 which transmit key information to the PDCS 70 for patient management to the physicians office or clinic. Information received by the PDCS 70 at the physicians office can be used to determine if the patient's status is satisfactory or whether an adjustment in diet or therapy is required in order to maintain the patient's health and to prevent worsening of status that may eventually lead to hospitalization. On a given day, only a small percentage of patients may present with a deteriorating condition and require follow-up by a health care practitioner. It therefore is advantageous to evaluate patient information automatically using an algorithm that identifies those patients that require follow-up and a potential change in therapy. Such an algorithm can identify patients that require follow-up by, for example, analyzing current data vs. preset limits determined by the physician (e.g. if LV EDP>15 mmHg, then trigger follow up), or analyzing the results of a mathematical model applied to a waveform or portion of a waveform such as the diastolic portion of the LV pressure signal.

### Description of the Implantable Telemetry Device

Referring to FIG. 2, the implantable telemetry device can include a telemetry unit (TU) 40, an electrical lead 50, and a remote sensing assembly (RSA) 30 (e.g., a pressure sensing device). The RSA 30 can include a body portion having transducing electronics (e.g., an electronics module 33) within a housing 32 that is shaped about a longitudinal axis. The housing 32 can have a coating thereon that promotes tissue growth to anchor the housing within a heart wall (e.g., the ventricular septum). The RSA 30 can also include a pressure transmitting port 28 (e.g., as part of a pressure transmitting catheter (PTC) 34) located at a distal longitudinal end of the housing 32 such that when the body portion is anchored in a heart wall (e.g., the ventricular septum) that port 28 transmits a pressure from a ventricle.

The TU 40 can include telemetry electronics (not visible) contained within housing 42. The TU housing 42 can protect the telemetry electronics from the harsh environment of the human body. The housing 42 can be fabricated of a suitable biocompatible material such as titanium or ceramic and can be hermetically sealed. The outer surface of the housing 42 can serve as an EGM sensing electrode. If a non-conductive material such as ceramic is used for the housing 42, conductive electrodes can be attached to the surface thereof to serve as EGM sensing electrodes. The housing 42 can be coupled to the lead 50 via a connector (not visible), and include an electrical feedthrough to facilitate connection of the telemetry electronics to the connector. The telemetry electronics disposed in the TU 40 can be the same or similar to those described in U.S. Pat. Nos. 4,846,191, 6,033,366, 6,296,615 or PCT Publication WO 00/16686, all to Brockway et al.

Still referring to FIG. 2, the flexible electrical lead 50 can connect the electronics module 33 and sensor housing 32 to the telemetry unit 40. The lead 50 can contain, for example, four conductors—one each for power, ground, control in, and data out. The lead 50 can incorporate conventional lead design aspects as used in the field of pacing and implantable defibrillator leads. The lead 50 can include a strain relief 52 at the connection to the proximal end of the sensor housing 32. The lead 50 can also include a connector which allows the RSA 30 to be connected and disconnected from the TU 40 in the surgical suite to facilitate ease of implantation. The lead 50 can optionally include one or more EGM electrodes.

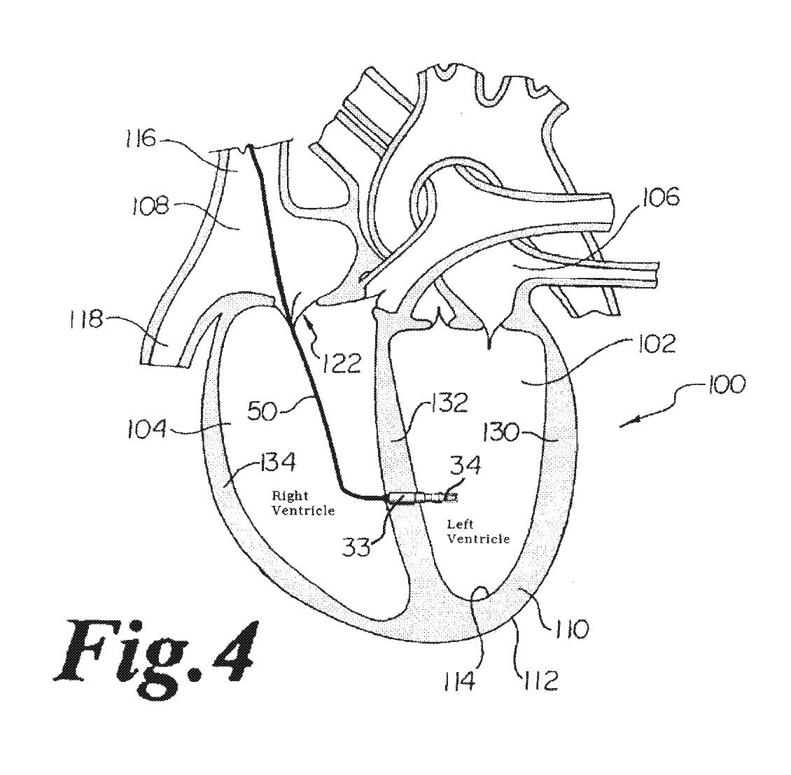

FIGS. 3A, 3B, and 3C depict a more detailed view of the remote sensor assembly (RSA) 30 shown in FIG. 2. The RSA 30 can include transducing electronics (e.g., a pressure transducer 31) within an electronics module 33 contained within a housing 32. The sensor housing 32 can protect the pressure transducer 31 and other electronics from the harsh environment of the human body. The housing 32 can be fabricated of a suitable biocompatible material such as titanium and can be hermetically sealed. The outer surface of the housing 32 can serve as an electrogram (EGM) sensing electrode. The proximal end of the housing 32 can include an electrical feedthrough to facilitate connection of the electronics module 33 in the housing 32 to a flexible lead 50. The distal bottom side of the housing can include a pressure transducer header to facilitate mounting of the pressure transducer 31 and to facilitate connection to a pressure transmission catheter (PTC) 34. The housing 32 can have a visible marking directly opposite the location of the PTC 34 such that the location of the PTC 34 can be visualized during surgery.

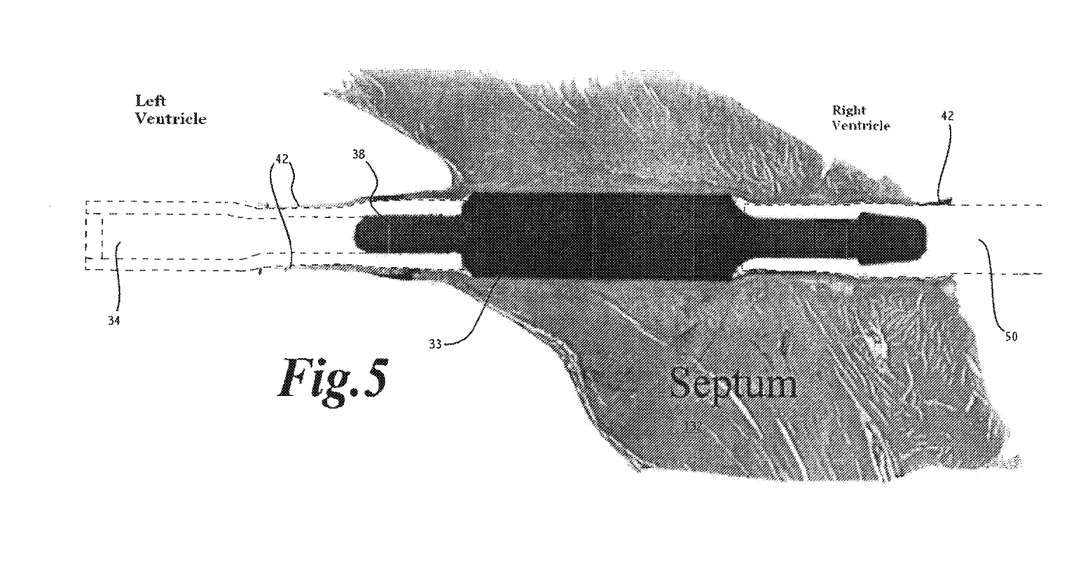

The housing 32 can be adapted for implantation into a heart wall (e.g., the ventricular septum 132). By implanting the housing 32 within a heart wall, the amount of volume taken up by the electronics module adjacent to a heart wall can be reduced. For example, by implanting the electronics module in the ventricular septum 132, this can reduce the amount of volume taken up by the implantable telemetry device within a ventricle (e.g., the right ventricle when positioning the PTC 34 within the left ventricle, as shown in FIG. 4) This can also reduce the contact area in the left ventricle. The outer surface of the housing 32 can be configured to anchor the electronics module 33 within a passage formed through a heart wall. In some implementations, the housing can include spikes, scales, or other protrusions. For example, fish scales can be angled towards the lead 50 to allow for relatively easy insertion into a passage in an advancing direction, but to provide substantial resistance to removal in the reverse direction. In some implementations, the housing can be anchored into a passage by friction between the housing 32 and the inside surface of a passage formed through a heart wall, without additional anchoring features.

The housing 32 can be adapted to allow for tissue growth from a heart wall around and/or into the housing 32 to further anchor the housing 32 into the heart wall. For example, the housing 32 can have a tissue in-growth promoting surface. In some implementations, the outside of the housing can include pores. The pores can be sized to allow tissue surrounding the housing (e.g., tissue from the ventricular septum 132) to grow into the pores and anchor the housing 32. In some implementations, the housing 32 can include a coating 37 that promotes tissue growth to anchor the housing within a heart wall (e.g., the ventricular septum). FIG. 3A shows an RSA 30 including a tissue growth promoting coating 37, while FIG. 3B shows the housing without a coating.

The coating 37 can be a thin-walled cover placed over housing 32. For example, coating 37 can include a thin-walled tube or sock (closed-ended) of open cell porous polymer. Coating 37 can promote tissue ingrowth (passivation) and reduce the risk of thromboemboli formation. For example, the controlled ingrowth of tissue into the ePTFE can also allow for an easier removal of the RSA 30 from the ventricular septum 132. For example, the coating 37 can include a thin walled tube of expanded fluoropolytetrafluoroethylene (ePTFE) or a woven tube of polyethylene terephthalate, (e.g., DACRON). A number of other materials can also be suitable for use in coating 37, for example fluoropolytetrafluoroethylene (PTFE), polyethylene (PE), polypropylene (PP), polyvinylchloride (PVC), and/or polyurethane. A number of manufacturing processes can be used to create coating 37. For example, coating 37 can be woven from a plurality of fibers. By way of a second example, coating 37 can be formed from one or more sections of shrink tubing. The shrink tubing sections can be positioned and then shrunk by the application of heat.

Referring again to FIG. 3A, coating 37 can extend along lead 50 and/or PTC 34. Coating 37 can, in some implementations, leave between 4 to 8 millimeters of the PTC 34 uncovered by the coating 37 (e.g., about 6 mm). In some implementations, coating 37 can cover portions of the RSA 30 that are implanted into the heart wall (e.g., the septum 132). In some implementations, the coating 37 can extend along a portion of the PTC 34, but not along the entire length of housing 32. In some implementations, a woven tube of polyethylene terephthalate, (e.g., DACRON) can overlie a portion of the housing 32 and a thin walled tube of ePTFE can overlie a portion of the PTC 34.

Referring to FIG. 31B, a pressure transducer 31 and other associated electronics can be disposed in an electronics module 33 surrounded by housing 32. The pressure transducer 31 can be of the piezoresistive, optical, resonant structure, or capacitive type. For example, the pressure transducer can include a piezoresistive wheatstone bridge type silicon strain gauge. Examples of suitable pressure transducers are disclosed in U.S. patent application Ser. No. 10/717,179, filed Nov. 17, 2003, entitled Implantable Pressure Sensors, the entire disclosure of which is incorporated herein by reference. The electronics in module 33 can provide excitation to the pressure transducer 31, amplify the pressure and EGM signals, and/or digitally code the pressure and EGM information for communication to the telemetry unit 40 via the flexible connecting lead 50. The signals from the electronics module 33 can be transmitted through lead 50 via electrical conductors 39. In some implementations, the electronics module 33 can include an application-specific integrated circuit (ASIC) 35 and/or a circuit substrate 36. The electronics module 33 can also provide for temperature compensation of the pressure transducer 31 and provide a calibrated pressure signal. Although not specifically shown, it can be useful to include a temperature measurement device within the electronic module to compensate the pressure signal from temperature variations. For example, the temperature measurement can select a look up table value to modify the pressure reading. This operation can be performed in any of the RSA 30, TU 40, or HDCS 60.

The PTC 34 transmits pressure from the pressure measurement site (e.g., LV) to the pressure transducer 31 located inside the sensor housing 32. The PTC 34 can include a tubular structure 22 including a proximal shaft portion and a distal shaft portion, with a liquid-filled lumen 24 extending therethrough to a distal opening or port 28. The PTC 34 can optionally include one or more EGM electrodes or other physiological sensors as described in U.S. Pat. No. 6,296,615 to Brockway et al.

The proximal end of the PTC 34 is connected to the pressure transducer 31 via a nipple tube 38, thus establishing a fluid path from the pressure transducer 31 to the distal end of the PTC 34. The proximal end of the PTC 34 can include an interlocking feature to secure the PTC 34 to the nipple tube of the pressure transducer 31. For example, the nipple tube 38 can have a knurled surface, raised rings or grooves, etc., and the proximal end of the PTC 34 can include an outer clamp, a silicone band, a spring coil or a shape memory metal (e.g., shape memory NiTi) ring to provide compression onto the nipple tube 38.

A barrier 26 such as a plug and/or membrane can be disposed in the port 28 to isolate the liquid-filled lumen 24 of the PTC 34 from bodily fluids, without impeding pressure transmission therethrough. If a gel (viscoelastic) plug 26 is utilized, one to several millimeters of a gel can be positioned into the port 28 at the distal end of the PTC 34. The gel plug 26 comes into contact with blood and transfers pressure changes in the blood allowing changes in blood pressure to be transmitted through the fluid-filled lumen 24 of the PTC 34 and measured by the pressure transducer 31. The gel plug 26 can be confined in the port 28 at the tip of the PTC 34 by the cohesive and adhesive properties of the gel and the interface with catheter materials. The chemistry of the gel plug 26 can be chosen to minimize the escape of the fluid in the remainder of the PTC 34 by permeating through the gel. In some embodiments, the fluid can be fluorinated silicone oil and the gel can be dimethyl silicone gel.

The gel plug 26 can have a high penetration value in order to inject the gel plug 26 into the port 28 at the tip of PTC 34, as well as to obtain accurate measurements. Penetration value is a measure of the “softness” of the gel by assessing the penetration of a weighted cone into the gel within a specified time. Also preferably, to meet in-vivo performance requirements for measuring blood pressure, the gel 26 can be soft enough to not induce hysteresis, but not so soft that significant washout occurs. Washout can also be reduced by choosing a gel that becomes fully cross-linked and has a low solubility fraction. Furthermore, a fully cross-linked gel can be very stable, and can thereby increase the usable life of the device. In some embodiments, the gel can also include a softener (e.g., dimethyl silicone oil). The gel plug 26 can be flush with the distal end of the PTC 34 or can be recessed (e.g., 0.5 mm) to shelter the gel plug 26 from physical contact and subsequent disruption that can occur during the procedure of insertion into the heart.

The pressure transmission fluid contained within the lumen 24 of the PTC 34 proximal of the barrier 26 can include a relatively low viscosity fluid and can be used to tune the frequency response of the PTC 34 by adjusting the viscosity of the transmission fluid. The pressure transmission fluid can include a relatively stable and heavy molecular weight fluid. The specific gravity of the transmission fluid can be low in order to minimize the effects of fluid head pressure that could result as the orientation of the PTC 34 changes relative to the sensor 31. The pressure transmission fluid can have minimal biological activity (in case of catheter or barrier failure), can have a low thermal coefficient of expansion, can be insoluble in barrier 26, can have a low specific gravity, can have a negligible rate of migration through the walls of PTC 34, and can have a low viscosity at body temperature. In some implementations, the pressure transmission fluid can incorporate end-group modifications (such as found in fluorinated silicone oil) to make the transmission fluid impermeable in the barrier material 26. In some implementations, the fluid can include a perfluorocarbon. Examples of suitable gels and transmission fluids can be found in U.S. Pat. No. 6,296,615 to Brockway et al.

Various other and specific embodiments of the PTC 34 can be found in U.S. Pat. Application No. 2005/0182330 A1 to Brockway et al. For example, the proximal and distal ends of the PTC 34 can be flared to have a larger inside diameter (ID) and outside diameter (OD), for different purposes. The distal end of the PTC 34 can be flared to provide a port 28 having a larger surface area as discussed above, and the proximal end of the PTC 34 can be flared to accommodate the nipple tube 38 and provide a compression fit thereon. The proximal flared portion can have an ID that is smaller than the nipple tube 38 to provide a compression fit that will be stable for the life of the RSA 30. The mid portion or stem of the PTC 34 can have a smaller ID/OD, with gradual transitions between the stem and the flared ends. The gradual transitions in diameter can provide gradual transitions in stiffness to thereby avoid stress concentration points, in addition to providing a more gradual funneling of the gel into the stem in the event of thermal retraction. The unitary construction of the PTC 34 can also provide a more robust and reliable construction than multiple piece constructions. Absent the gradual transitions, the PTC 34 can be more susceptible to stress concentration points, and the gel and the transmission fluid are more likely to become intermixed and can potentially dampen pressure transmission. By way of example, not limitation, the proximal flared portion can have an ID of 0.026 inches, an OD of 0.055 inches, and a length of about 7 mm. The stem (mid) portion can have an ID of 0.015 inches, and OD of 0.045 inches, and a length of about 7 mm. The distal flared portion can have an ID of 0.035 inches, an OD of 0.055 inches, and a length of about 4 to 5 mm. The proximal taper can have a length of about 0.5 mm and the distal taper can have a length of about 1.25 mm. The gel plug 26 can have a length of about 3 mm and resides in the distal flared portion. In some implementations, (e.g., where a relatively short PTC 34 is utilized) the fluid-filled lumen 24 of the PTC 34 can be completely filled with the barrier material 26 (e.g., gel). In combination with the gel plug 26, or in place thereof, a thin membrane can be disposed over the port 28.

The PTC 34 can have a length that provides adequate access across the heart wall (e.g., septum or myocardium) and into the heart chamber (e.g., LV) while being as short as possible to minimize head height effects associated with the fluid-filled lumen 24. The PTC 34 may be straight or may be curved, depending on the particular orientation of the RSA 30 relative to the heart wall and the chamber defined therein at the insertion point. The PTC 34 can have a length sufficient to allow the port 28 of the PTC 34 to reside within a chamber of the heart 100 without the heart wall tissue to propagate to overcoat the port 28. In some implementations, the PTC 34 can be between 1 cm and 2.5 cm in length (e.g., about 2 cm in length). As discussed above, coating 37 can also overlie a portion of the PTC 34 (e.g., the distal portion). In some implementations, the proximal portion of the PTC 34 can be ovennolded with silicone to provide stress relief, flex fatigue strength, and a compliance matching mechanism at the entrance to the myocardium.

### Description of Implantation Process

To facilitate a discussion of the implantation process, it is helpful to define and label some of the anatomical features of the heart 100 shown in FIG. 4. The heart 100 includes four chambers, including the left ventricle (LV) 102, the right ventricle (RV) 104, the left atrium (LA) 106, and the right atrium (RA) 108. The LV 102 is defined in part by LV wall 130, the RV 104 is defined in part by RV wall 134, and the LV 102 and the RV 104 are separated by ventricular septum 132.

The right atrium 108 receives oxygen deprived blood returning from the venous vasculature through the superior vena cava 116 and inferior vena cava 118. The right atrium 108 pumps blood into the right ventricle 104 through tricuspid valve 122. The right ventricle 104 pumps blood through the pulmonary valve and into the pulmonary artery which carries the blood to the lungs. After receiving oxygen in the lungs, the blood is returned to the left atrium 106 through the pulmonary veins. The left atrium 106 pumps oxygenated blood through the mitral valve and into the left ventricle 102. The oxygenated blood in the left ventricle 102 is then pumped through the aortic valve, into the aorta, and throughout the body via the arterial vasculature.

Referring to FIGS. 3C and 4, the RSA 30 can be implanted through a heart wall such that the distal end of the PTC 34 resides in the LV 102, the RV 104, or any other chamber of the heart 100. For example, the PTC 34 can be positioned across the ventricular septum 132 such that the pressure transmitting port 28 of the PTC 34 is disposed in the LV 102. As shown in FIG. 4, an LV endocardial pressure can be measured via the PTC 34, which transmits blood pressure from within the LV 102 to the pressure sensor contained in the housing 32. The pressure sensor (or pressure transducer) 31, together with the associated electronics in the housing 32, convert the pressure signal into an electrical signal (analog or digital) which is transmitted to the TU 40 via lead 50.

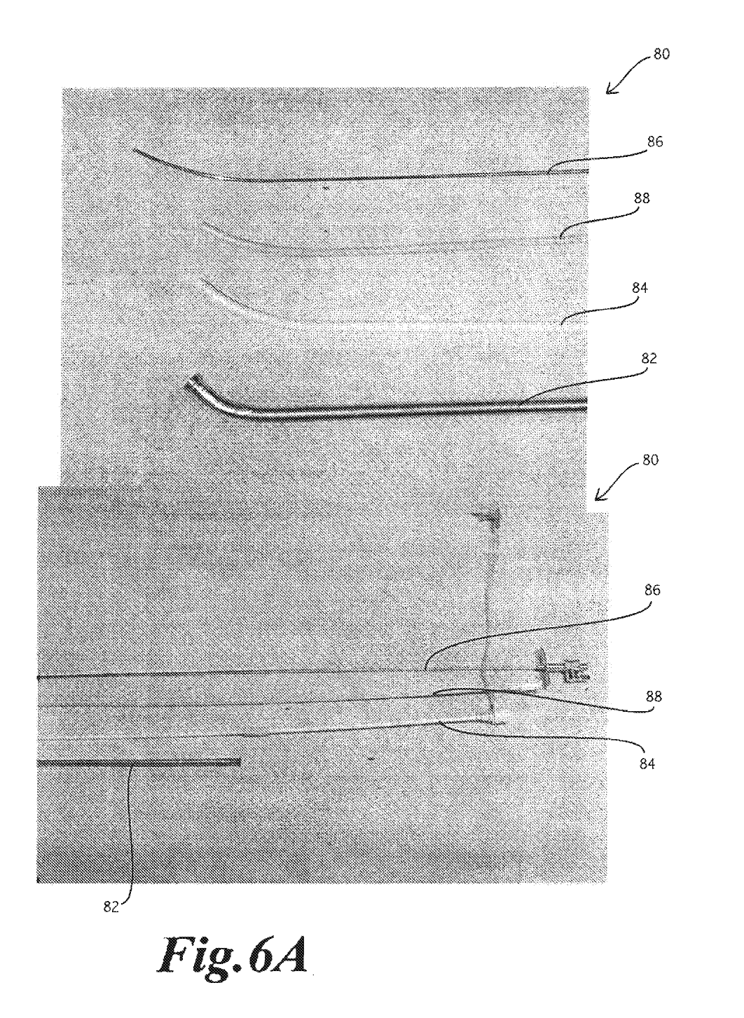

FIG. 5 is a picture of the RSA inserted through a ventricular septum 132 of a sheep's heart after the heart has responded and healed (e.g., after 28 days). Because the picture uses an imaging technique which does not pick up polymeric portions of the RSA, the electrical lead 50 and the PTC 34 are shown with dotted lines. To obtain the image shown, the cardiac septum 132 containing the RSA 30 was embedded in Technovit 7200 resin, sectioned, ground, stained with toluidine blue, and evaluated microscopically. FIG. 5 clearly shows the growth of tissue 42 along an ePTFE coating on lead 50 and the PTC 34. The tissue growth has stopped growing and ends before it reaches the end of the PTC 34, enabling the port 28 of the PTC 34 to remain unobstructed by tissue. The presence of coating 37 (e.g., ePTFE) can allow the tissue surrounding the passage to resolve (cease to proliferate). In some implementations, the tissue will resolve within 28 days. As shown in FIG. 5, the tissue surrounding the housing 32 of the electronics module 33 can tightly conform to the housing.

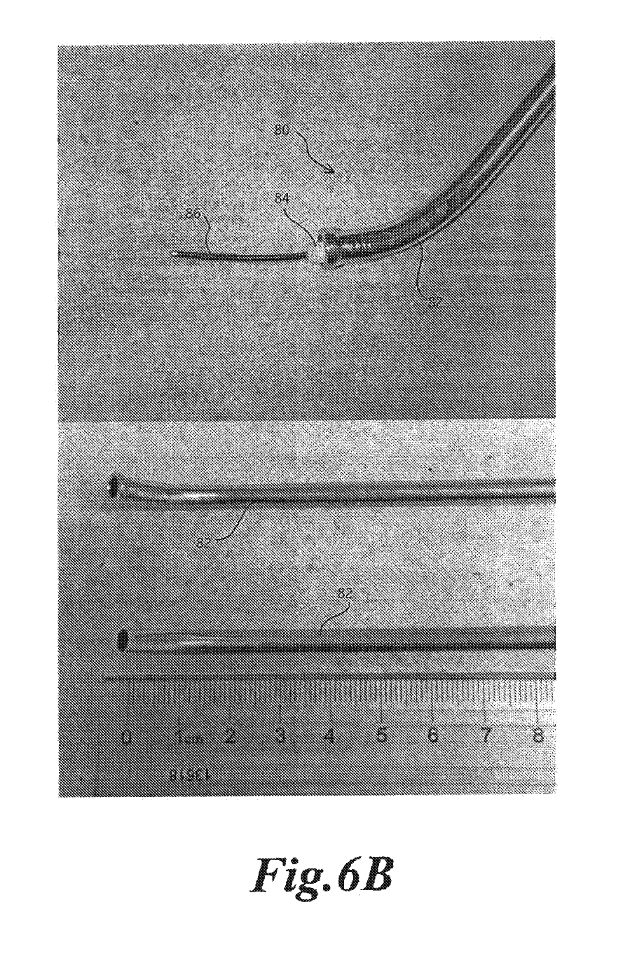

The RSA 30 can be implanted by a number of techniques. For example, the RSA 30 can be implanted by an assembled introducing apparatus 80, including an introducer 82, a sheath 84 positioned within the introducer 82, and a needle 86 positioned within the sheath 84. FIGS. 6A and 6B show the parts of the introducing apparatus 80. In some implementations, the assembled introducing apparatus 80 can also include a centering tube 88 disposed around the needle 86, to center the needle within the sheath 84. The needle 86 can be a modified Brockenbrough needle. The needle can be hollow tipped. For example, the RSA 30 can be implanted into a ventricular septum 132, as shown in FIG. 4, by performing the following steps: (a) accessing a vein such as the subcalavian or jugular; (b) advancing the assembled introducing apparatus 80 into the vein; (c) advancing the introducing apparatus 80 to the RV 104; (d) placing the distal end of the introducer 82 against the ventricular septum 132; (e) forming an initial passage through the ventricular septum by extending the needle 86 out of the introducing apparatus, through the ventricular septum 132, and into the LV 102; (f) extending a sheath 84 from within the introducing apparatus into the ventricular septum 132 for registration; (g) removing the needle 86 and the centering tube 88 from within the sheath 84 and the introducer 82; (h) inserting the RSA 30 and lead 50 through the sheath 84 into the passage formed by the needle such that the housing 32 of the electronics module 32 resides in the passage and the distal end of the PTC 34 resides in the LV 102; (i) removing the sheath 84 from the ventricular septum 132; and (j) removing the introducer 82 while not dislodging the RSA 30 from the passage.

The introducing apparatus 80 can be guided to the RV 104 by a guidance scheme. For example, fluoroscopy can be used to help guide the introducing apparatus 80. The use of pressure measurements at the distal tip of the introducing apparatus can also help determine the location of the distal tip of the introducing apparatus 80 by monitoring the pressure changes (e.g., the RV 104, the ventricular septum 132). By monitoring changes in pressure at the tip of the introducing apparatus 80, the location of the tip can be determined. The use of fluoroscopy can further assist in determining the positioning of the tip of the introducing apparatus 80. The monitoring of pressure changes at the distal tip of the introducing apparatus 80 can also allow a user to confirm a location of a distal tip of the needle while extending the needle through the ventricular septum into the left ventricle during initial registration.

Furthermore, by monitoring the pressure changes detected by the RSA 30 during implantation, the location of the RSA 30 can be confirmed while inserting the RSA 30 through the interior of the sheath into the ventricular septum. Furthermore, pressure changes can also help to measure the width of the ventricular septum 132 and insure proper placement of the RSA 30 within the ventricular septum 132. For example, as the RSA 30 is introduced through the passage from the RV 104 into the LV 102, the PTC 34 can detect a pressure change of about 50 to 100 mmHg. This pressure change can indicate that port 28 of the PTC 34 is positioned within the LV 102. Fluoroscopy and device marking can also be used to confirm the depth and placement of the RSA within the ventricular septum 132.

The needle 86 can be a modified Brockenbrough needle. The needle 86 can have a smaller diameter than the housing 32 of the electronics module 33. The passage formed by the needle 86 can have a diameter smaller than the housing 32. Accordingly, the insertion of the RSA 30 though the passage can further stretch the passage and result in the final dilatation of the passage. By sizing the needle 86 to produce a passage having a smaller diameter than the housing 32, the implantation of the housing within the ventricular septum 132 can ensure a frictional anchoring of the housing 32 within the passage of the ventricular septum 132. Once the PTC 34 resides in the LV 102 and the housing 32 is frictionally anchored in the ventricular septum 132, the sheath 84 and the introducer 82 can be removed without dislodging the RSA 30.

The RSA 30 can also allow for easier removal of the RSA 30 from within the ventricular septum 132. The presence of the coating, e.g., ePTFE, on the outside of the housing 32 can allow for a controlled ingrowth of tissue such that the tissue surrounding the RSA 30 still allows for removal of the RSA 30 without causing significant damage to the ventricular septum 132. Furthermore, the shape of the RSA 30 can allow for the RSA 30 to slip out of the passage formed through the ventricular septum 132. Furthermore, in some implementations, the RSA 30 can be free of anchoring devices that would prevent the RSA 30 from being able to slip out of the passage, such as spikes that would lock the RSA 30 into the ventricular septum 132 or self-expanding portions that would expand in the left ventricle (LV) to lock the RSA 30 into the heart. The use of spikes or self-expanding portions could require the use of invasive heart surgery to remove the RSA 30 from the heart.

The entire disclosure of all patents and patent applications mentioned in this document are hereby incorporated by reference herein.

A number of implementations have been described. Nevertheless, it will be understood that various modifications may be made without departing from the spirit and scope of this disclosure. For example, the electrical lead 50 can, in some implementations, be connected directly to a device outside of the body rather than to an internally implanted telemetry unit (TU) 40. Accordingly, other embodiments are within the scope of the following claims.

What is claimed is:

1. A pressure sensing device comprising:
a body portion having transducing electronics within a housing that is shaped about a longitudinal axis, the housing having a coating thereon that promotes tissue growth to anchor the housing within a ventricular septum;
a pressure transmitting port located at a distal longitudinal end of the body portion such that a ventricle pressure being sensed is transmitted through the port and to the transducing electronics when the body portion is anchored in the ventricular septum; and
an electrical lead connected to the transducing electronics and exiting from a proximal longitudinal end of the body portion.

2. The pressure measurement device of claim 1, wherein the coating comprises pores.

3. The pressure measurement device of claim 2, wherein the coating promotes tissue ingrowth of the ventricular septum into the pores to anchor the body portion in the ventricular septum.

4. The pressure measurement device of claim 1, wherein the coating comprises expanded polytetrafluoroethylene.

5. The pressure measurement device of claim 1, wherein the coating comprises polyethylene terephthalate.

6. A method of measuring pressure in a left ventricle, the method comprising:
inserting a pressure sensing device through a ventricular septum to sense a pressure in a left ventricle, the pressure sensing device including:
(a) a body portion having transducing electronics within a housing that is shaped about a longitudinal axis, the housing having a coating thereon that promotes tissue growth to anchor the housing within the ventricular septum,
(b) a pressure transmitting port located at a distal longitudinal end of the body portion such that a ventricle pressure being sensed is transmitted through the port and to the transducing electronics when the body portion is anchored in the ventricular septum, and
(c) an electrical lead connected to the transducing electronics and exiting from a proximal longitudinal end of the body portion;
wherein inserting the pressure sensing device includes positioning the body portion in the ventricular septum and the port in the left ventricle.

7. The method of claim 6, wherein the coating comprises pores.

8. The method of claim 7, wherein the coating promotes tissue ingrowth of the ventricular septum into the pores to anchor the body portion in the ventricular septum.

9. The method of claim 6, wherein the coating comprises expanded polytetrafluoroethylene.

10. The method of claim 6, wherein the coating comprises polyethylene terephthalate.

11. The method of claim 6, wherein the body portion is anchored in the ventricular septum by frictional engagement between the coating and the ventricular septum.

12. The method of claim 6, further comprising forming a passage in the ventricular septum, wherein inserting the pressure sensing device includes passing the passing the pressure sensing device through the passage.

13. The method of claim 12, wherein the passage has a first diameter and the housing has a second diameter greater than the first diameter, wherein inserting the body portion in the ventricular septum results in the final dilatation of the passage.

14. The method of claim 6, further comprising:
inserting an introducing apparatus into a vein, the introducing apparatus including an introducer, a sheath disposed at least partially in the introducer, a centering tube disposed at least partially in the sheath, and a needle disposed at least partially in the centering tube, wherein the centering tube centers the needle with respect to the sheath;
advancing the introducing apparatus to a right ventricle;
placing a distal end of the introducing apparatus against the ventricular septum;
extending the needle through the ventricular septum into a left ventricle for initial registration;
extending the sheath partially into the ventricular septum for registration;
removing the needle and the centering tube and leaving the sheath in place to maintain registration;
passing the pressure sensing device through an interior of the sheath to insert the pressure sensing device through the ventricular septum; and
removing the sheath and the introducer without dislodging the pressure sensing device.

15. The method of claim 14, further comprising using a pressure measurement at the distal tip of the introducing apparatus to determine a location of the distal tip of the introducing apparatus while advancing the introducing apparatus to the right ventricle based on pressure changes from one location to another location while advancing the introducing apparatus to the right ventricle.

16. The method of claim 15, further comprising using fluoroscopy to determine a location of the distal tip of the introducing apparatus while advancing the introducing apparatus to the right ventricle.

17. The method of claim 14, further comprising:
confirming a location of a distal tip of the needle while extending the needle through the ventricular septum into the left ventricle for initial registration; and
confirming a location of the pressure sensing device while inserting the pressure sensing device through the interior of the sheath into the ventricular septum.

18. The method of claim 14, wherein the needle is a modified Brockenbrough needle.

19. The method of claim 14, wherein the introducing apparatus is assembled prior to being inserted into the vein, so that a distal end of the sheath protrudes slightly through a flared distal end of the introducer, and a distal end of the needle extends through a distal end of the centering tube and the distal end of the sheath.

20. The method of claim 14, wherein the centering tube has an outer diameter substantially equal to an outer diameter of the pressure measurement device and an inner diameter substantially equal to an outer diameter of the needle.
